(19) United States
(12) Patent Application Publication
(10) Pub. No.: US 2025/0195775 A1
(43) Pub. Date: Jun. 19, 2025

(54) CARTRIDGE HOLDER AND CARTRIDGE ASSEMBLY FOR AN INJECTION DEVICE

(71) Applicant: Sanofi, Paris (FR)

(72) Inventors: Michael Helmer, Frankfurt am Main (DE); Reinhard Rieger, Obertshausen (DE); Martin Vitt, Frankfurt am Main (DE)

(21) Appl. No.: 18/847,916

(22) PCT Filed: Mar. 15, 2023
(86) PCT No.: PCT/EP2023/056533

§ 371 Date: Sep. 17, 2024

(30) Foreign Application Priority Data
Mar. 17, 2022 (EP) 22315068.1

(51) Int. Cl. A61M 5/24

(52) U.S. Cl. CPC A61M 5/2422; A61M 2005/2411; A61M 2005/244

(57) ABSTRACT

The present disclosure relates to a cartridge assembly and a cartridge holder for an injection device, wherein the cartridge holder comprises:
an elongated body extending in a longitudinal direction (z), the elongated body comprises a distal end and a proximal end opposite to the distal end, wherein the elongated body confines a cartridge receiving space to accommodate a medicament container,
a retainer part comprising a retainer sidewall confining a distal portion of the cartridge receiving space, the retainer part comprising a proximal end,
a connector part comprising a connector sidewall and a distal connector configured to connect to the proximal end of the retainer part and comprising a proximal connector configured to connect to a distal end of a device body of the injection device, wherein the device body is configured to accommodate a drive mechanism to operably engage with the medicament container.

### CROSS-REFERENCE TO RELATED APPLICATIONS

This is a United States National Stage Application, filed under 35 U.S.C. § 371, of International Patent Application No. PCT/EP2023/056533, filed on Mar. 15, 2023, which claims priority to European Patent Application No. 22315068.1, filed on Mar. 17, 2022, the disclosures of which are incorporated herein by reference in their entireties.

### TECHNICAL FIELD

The present disclosure relates to a cartridge holder, a cartridge assembly and a container assembly for an injection device. The disclosure further relates to an injection device and to a method of assembling an injection device.

### BACKGROUND

Drug delivery devices allowing for multiple dosing of a required dosage of a liquid medicinal product and further providing administration of such liquid drug to a patient, are as such well known in the prior art. Generally, such devices have substantially the same purpose as that of an ordinary syringe. Typically, a medicinal product to be administered is provided in a cartridge having a moveable piston or bung mechanically interacting with a piston rod of a drive mechanism of the drug delivery device. By applying thrust to the piston, a certain and pre-defined amount of the medicinal fluid is expelled from the cartridge.

A patient suffering from a particular disease may require a certain amount of a medicament to either be injected via a pen-type injection syringe or infused via a pump. With respect to reusable injection or delivery devices, a patient may have to load or to replace a cartridge. Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect medicament into a drug delivery device and, then, dispense said medicament (such as a rapid or basal type of insulin) without being aware that the medical delivery device was perhaps neither designed nor intended to be used with such a cartridge.

As such, there is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device.

There is therefore a general need to physically dedicate or mechanically code a cartridge and/or cartridge holder to its drug type and design an injection device that only accepts or works with the dedication or coded features provided on or with the cartridge and/or cartridge holder so as to prevent unwanted cartridge cross use. For this it is generally conceivable to provide a dedicated cartridge assembly, i.e. a cartridge holder provided with a cartridge or medicament container readily assembled therein. Here, the cartridge may be non-detachably fixed inside the cartridge holder. The cartridge holder may be individually dedicated or physically encoded in accordance to the particular type of a cartridge and/or to a particular medicament contained in such a cartridge or medicament container.

For reusable injection devices and upon exhaustion of the medicament, the cartridge or medicament container has to be replaced. With cartridge assemblies comprising a cartridge holder with a cartridge fixed therein it may be intended to discard the entire cartridge assembly and to replace the cartridge assembly by a new one. Here, and on the one hand, the disposal of the entirety of the cartridge holder may have a negative impact on the ecological footprint of the injection device. On the other, hand, for preventing unintended cross-use of a cartridge with a wrong injection device or drive mechanism, it is desirable to irreversibly mechanically dedicate or to irreversibly connect the cartridge with a correspondingly encoded cartridge holder.

Hence, there is a general need for a dedicated cartridge that allows the medical delivery device to be used with only an authorized cartridge containing a specific medicament while also preventing undesired cartridge cross use.

With drug delivery devices comprising a multi-component housing, e.g. having a first and a second housing component, it is also desirable to provide a failure safe and well-defined mechanical connection between the housing components, which may be detachably or non-detachably connectable.

### SUMMARY

In one aspect the present disclosure relates to a cartridge holder for an injection device, such as an injection pen. The cartridge holder comprises an elongated body extending in a longitudinal direction. The elongated body comprises a distal end and a proximal end. The proximal end is located opposite to the distal end. The elongated body confining a cartridge receiving space to accommodate a medicament container, typically implemented as a cartridge. The cartridge holder further comprises a retainer part comprising a retainer sidewall confining a distal portion of the cartridge receiving space. The retainer part comprises a proximal end. The cartridge holder further comprises a connector part comprising a connector sidewall and a distal connector. The distal connector is configured to mechanically connect to the proximal end of the retainer part. The connector part further comprises a proximal connector configured to connect to a distal end of a device body of the injection device. The device body is configured to accommodate or to receive a drive mechanism. The drive mechanism is configured to operably engage with the medicament container.

With some examples the cartridge holder constitutes or forms a distal housing component of the injection device whereas the body constitutes or forms a proximal housing component of the injection device. With some examples the device body and the cartridge holder are detachably or releasably connectable. With some examples the injection device is implemented as a reusable injection device. Here, the cartridge holder can be detached from the device body for exchanging the medicament container.

With some examples of the cartridge holder the retainer part is a reusable component and the connector part is a disposable component. Here, and for exchanging a medicament container it may be intended or necessary to disconnect the retainer part from the connector part in order to provide access to the medicament container.

With some examples the proximal connector of the connector part is mechanically encoded in accordance to the medicament container and/or in accordance to the medicament provided inside the medicament container. A mechanical encoding of the proximal connector enables a mechanical connection of the cartridge holder to a matching or dedicated device body provided and equipped with a suitable drive mechanism. This way, a non-intended cross use of a cartridge holder with a non-matching device body or non-matching drive mechanism can be effectively prevented. Accordingly, patient safety can be enhanced and misuse of the injection device, i.e. an improper or non-intended connection of a cartridge holder to a non-matching device body can be effectively avoided.

Moreover, the connector part may comprise or provide a mechanical adapter for attaching the retainer part to different types of device bodies. Hence, there may be provided differently configured connector parts each of which comprising an identical distal connector for connecting to a single type of or to identically shaped retainer parts.

With a set or kit of differently configured connector parts the proximal connectors of the respective connector parts may distinguish from each other. Hence, a first connector part of a kit or set of connector parts is provided with a first type of a distal connector and with a first type of a proximal connector. A second connector part is provided with the same first type of a distal connector but with a second type of a proximal connector. The first and the second proximal connectors distinguish from each other. The first proximal connector is connectable to a first type of a device body and is not connectable to a second type of the device body. Vice versa, the second type of the proximal connector is connectable to the second type of a device body but is incompatible with the first type of the device body. Non-matching pairs of the proximal connector and the device body are inoperable to mutually connect.

In this way, the cartridge holder can be mechanically encoded by selecting a suitable connector part matching with the respective medicament container. Insofar, there can be provided a container assembly, i.e. a matching pair of a connector part and a medicament container. Such a container assembly may be commercially distributed by a manufacturer of the medicament container. The container assembly may be connectable to one and the same type of a retainer part, which is a reusable part. Such a reusable retainer part can be sequentially used with a series of container assemblies.

The retainer part may be also universally usable and connectable to different types of container assemblies, each of which being dedicated and configured for a mechanical connection to only one specific device body equipped with a respective drive mechanism.

The presently proposed cartridge holder provides the benefit to reuse the retainer part for a large variety of differently configured connector parts and hence for differently configured device bodies of the same or different types of injection devices. In this way, production costs for the retainer part can be decreased. Moreover, an ecological footprint of the cartridge holder and hence of the entire injection device can be reduced.

By only or exclusively implementing the connector part as a disposable component the total number of reusable components or parts of the injection device can be increased compared to a solution of a dedicated or encoded cartridge holder, which in its entirety is configured or intended to be disposed after use when the cartridge is immovably fixed therein.

According to a further example the retainer part and hence the retainer sidewall confines a major portion of the cartridge receiving space. A minor portion of the cartridge receiving space may be confined by the connector part. Hence, both the retainer part and the connector part may contribute to the enclosure that confines the cartridge receiving space. The cartridge receiving space is defined by the volume inside the cartridge holder, which is entirely occupied by the medicament container, e.g. implemented as a cartridge.

Hence, the connector sidewall and the retainer sidewall adjoin in a longitudinal direction. They may be arranged in a partially nested or overlapping configuration such that at least a portion of the connector sidewall and at least a portion of the retainer sidewall exclusively form or contribute to the confinement of the cartridge receiving space.

According to a further example the connector sidewall forms a longitudinal extension of the retainer sidewall when the connector part is connected to the retainer part. Here, the connector sidewall and the retainer sidewall may flush in longitudinal direction. In particular, an inside surface of the connector sidewall may flush with an inside surface of the retainer sidewall. Also, an outside surface of the connector sidewall may flush with a respective outside surface of the retainer sidewall.

With some examples the connector part is located longitudinally between the retainer part and the device body. Hence, the retainer part and the device body do not mechanically engage. They are arranged longitudinally offset from each other. A longitudinal gap between the retainer part and the device body is filled or occupied by the connector part. In other words, the retainer part is exclusively connectable to the connector part. The retainer part is indirectly connectable to the device body only by the connector part. In this way the connector part provides a mechanical adapter for fixing and connecting the retainer part to the device body.

According to a further example the connector sidewall of the connector part confines a proximal portion of the cartridge receiving space. Here, the connector sidewall may confine the outside circumference of the, e.g. tubular shaped, cartridge receiving space. With further examples the connector sidewall or the connector part may also confine the cartridge receiving space in longitudinal direction.

According to a further example the connector part comprises a flange portion extending radially inwardly from the connector sidewall. The flange portion comprises a distally facing stop face configured to abut with a proximal end of the medicament container. Typically, the medicament container, e.g. implemented as a cartridge, comprises a tubular-shaped barrel. The tubular-shaped barrel may be made of a vitreous material, such as glass. The proximal end of the barrel of the medicament container may get in longitudinal abutment with the distally facing stop face of the flange portion protruding or extending radially inwardly from the connector sidewall. In this way, the connector provides a twofold or double function. On the one hand it provides a mechanical connection between the device body and the retainer part of the cartridge holder. On the other hand the connector part provides a longitudinal fixing of the medicament container inside the cartridge receiving space.

Typically, and with further examples the retainer part comprises a proximally facing stop face, e.g. provided at a distally located end wall of the retainer part or provided in the region of a narrowing shoulder portion of the retainer sidewall to engage with a complementary shaped distally facing end face or shoulder portion of the barrel of the medicament container. In this way and by mechanically attaching the connector part with the retainer part, e.g. by establishing a mechanical connection between the distal connector of the connector part with the proximal end of the retainer part the medicament container located inside the cartridge receiving space can be fixed with regards to the longitudinal direction.

Typically, and with some examples the medicament container can be clamped or at least confined in longitudinal direction between the proximally facing stop face of the retainer part and the distally facing stop face of the flange portion of the connector part when the distal connector of the connector part engages the proximal end of the retainer part. In this way, there can be provided a longitudinal fixing of the medicament container inside the cartridge receiving space, both with regard to the distal direction and with regards to the proximal direction.

According to a further example the distal connector of the connector part and the proximal end of the retainer part are configured to form at least one of a tension stable and a pressure stable mechanical connection. When providing a tension stable mechanical connection any distally directed tensile force applied to the cartridge holder can be transferred via the connector part into the device body. Hence, a tensile force applied in distal direction to the cartridge holder can be unalterably transferred and directed into or onto the connector part, which due to its tension stable and/or pressure stable connection with the device body may equally transfer to the device body.

With a pressure stable mechanical connection any proximally directed force applied to the cartridge holder unalterably transfers into or to the connector part. The respective proximally directed thrust or force effect can then unalterably transfer into or to the device body of the injection device. With a mechanical connection stable with regard to tension and pressure as seen in longitudinal direction the retainer part and the connector part can be mutually connected in a rather rigid and stiff manner. This provides a rather stable and long-lasting mutual connection not only between the retainer part and the connector part are also between the retainer part and the device body. The mutual connection between the connector part and the retainer part may be free of mechanical play. In this way, the user of the injection device may gain the impression that the cartridge holder, which is made of the retainer part and the connector part is a single or unitary component.

According to a further example the elongated body of the cartridge holder is formed by a mutual connection of the retainer part and the connector part. With some examples the retainer part and the connector part constitute the elongated body of the cartridge holder. The cartridge holder may consist of the retainer part and the connector part being mutually attached and fixed to each other.

According to a further example the connector part comprises a receptacle to receive a proximal end of the medicament container. Here, the connector sidewall may constitute or provide at least a portion of the confinement of the cartridge receiving space. In this way and when disconnecting the retainer part and the connector part at least a proximal portion of the medicament container may protrude longitudinally, e.g. in proximal direction from the proximal end of the retainer part. This enables and facilitates removal of the medicament container from the e.g. reusable retainer part.

With some examples the longitudinal extent of the retainer part is larger than the longitudinal extent of the connector part. The longitudinal extent of the retainer part may be twice as large as the longitudinal extent of the connector part. It may be 3 times as large as the longitudinal extent of the connector part. With further examples the longitudinal extent of the retainer part is 4 times larger, 5 larger or even 6 times larger than the longitudinal extent of the connector part.

In this way a fraction of a reusable part or portion of the cartridge holder can be increased compared to the fraction of a disposable part of portion of the cartridge holder, thus improving the ecological footprint of the cartridge holder and hence of the injection device.

According to a further example the connector part comprises a container fastener configured to mechanically engage with a proximal end of the medicament container. By way of the container fastener the connector part can be detachably or undetachably connected with the medicament container. The connector part fixed or attached to the medicament container may comprise or constitute a container assembly. The connector part, which may be mechanically encoded in accordance to the medicament container and/or in accordance to the medicament contained inside the medicament container can be used to characterize the medicament container. The connector part may be connectable with any of the available retainer parts so as to form a cartridge holder.

The proximal connector of the connector part and/or the proximal end of the connector part may be mechanically encoded so as to prevent unauthorized or non-intended connection with a device body of an injection device equipped with a non-matching drive mechanism. The container fastener may be implemented in many different ways. With some examples the container fastener provides a friction fit with the medicament container, e.g. with an outside surface of the barrel of the medicament container. With other examples the container fastener may comprise or provide a glued or welded connection with the barrel of the medicament container. With further examples there may be provided a form closure or positive fitting between the container fastener and the medicament container.

According to a further example the container fastener is configured to non-detachably connect to the medicament container. It may be clamped, glued or welded with the medicament container. A user attempting to disconnect the container fastener and hence the connector part from the medicament container may end up with destroying at least one of the medicament container and the connector part. In this way, unauthorized use of the cartridge holder and/or of the medicament container can be effectively avoided. Counterfeiting of the cartridge holder and/or unintended or unauthorized refilling or substitution of the cartridge holder with a non-authorized medicament container can be effectively prevented.

According to a further example the container fastener comprises a first fastening rib and a second fastening rib on opposite sides of the cartridge receiving space. The first fastening rib and the second fastening rib are configured to frictionally engage with the barrel of the medicament container. Here, the fastening rib may be made of a friction enhancing material. They may comprise a friction surface facing towards or extending into the cartridge receiving space. The fastening rib may be made of an elastic material, such as an injection moldable plastic material and/or of an elastomeric material providing a frictional locking between the fastening ribs and the barrel of the medicament container once the medicament container has been e.g. inserted into the receptacle formed by the connector sidewall of the connector part.

With some examples the first fastening rib and/or the second fastening rib may protrude radially inwardly into the cartridge receiving space such that a medicament container can only be inserted into the cartridge receiving face by a radially outwardly directed elastic deformation of the first fastening rib and/or of the second fastening rib. In this way there can be provided a rather stable and efficient frictional locking between the fastening ribs and the barrel of the medicament container.

According to a further example at least one of the first fastening rib and the second fastening rib protruding distally from the distal end face of the connector part engages with a complementary-shaped recess of the retainer part when the connector part is connected to the retainer part. Distally protruding fastening ribs may provide a guiding function in order to establish a mechanical connection between the distal connector of the connector part and the proximal end of the retainer part.

With some examples also the distal connector may protrude distally from the distal end of the connector part and/or from the connector sidewall. Here, the fastening rib may even protrude distally from the distal connector and may therefore provide a proper alignment as seen in circumferential direction to enable a smooth and easy connection of the retainer part and the connector part.

Insofar the first and second fastening ribs may also provide the function of guiding ribs for establishing a correct alignment of the retainer part and the connector part in the course of a mutual assembly of the retainer part and the connector part.

According to a further example one of the proximal end and the distal connector comprises a fastening element complementary shaped to a counter fastening element of the other one of the proximal end and the distal connector. Insofar, the proximal end may be provided with the fastening element and the distal connector may be provided with a complementary-shaped counter fastening element so as to provide e.g. a releasable connection between the retainer part and the connector part.

According to a further example one of the fastening element and the counter fastening element comprises a recess. The other one of the fastening element and the counter fastening element comprises a catch element configured to engage with the recess. With some examples the recess is provided in a sidewall of one of the retainer part and the connector part. Then, the catch element may be provided on or may protrude from a sidewall of the other one of the retainer part and the connector part.

The recess may be provided at the proximal end of the retainer sidewall. It may be accessible or may be provided on an inside portion of the retainer sidewall. The catch element may be provided on an outside facing portion of the connector sidewall. Here, the proximal end of the retainer part may comprise a receptacle to receive at least a complementary shaped insert section of the distal end of the connector sidewall. In this way an at least partially overlapping or nested configuration of the retainer part and the connector part can be provided, thereby providing a rather stable and rigid mutual connection between the retainer part and the connector part.

According to a further example the catch element of one of the retainer part and the connector part extends in longitudinal direction and is configured to engage with the recess provided in the connector sidewall or retainer sidewall of the other one of the retainer part and the connector part. According to a further example the catch element is deformable or movable in a radial direction to engage and/or disengage from the recess. Here, the radial direction extends substantially perpendicular to the longitudinal direction (z) as defined by the elongated shape of the elongated body or of the cartridge holder.

With further examples the catch element may protrude distally from the connector sidewall. Here, the connector sidewall may comprise a distally facing stop face complementary shaped to a proximally facing end face of the retainer sidewall. The catch element or several catch elements may protrude distally from an inside of the connector sidewall of the connector part and may engage with the recess provided on the inside of the retainer sidewall when the proximal end face of the retainer sidewall engages or abuts with the distal end face of the connector sidewall.

A longitudinal extension of at least one or several catch elements from an inside surface of the connector sidewall provides the benefit that the respective counter fastening element is effectively invisible from outside the cartridge holder when the retainer part and the connector part are mutually assembled. With further examples it is conceivable, that the connector part is provided with a recess in a connector sidewall and that the retainer part is provided with a longitudinally extending, e.g. longitudinally protruding catch element to engage with the respective recess.

With some examples the recess in the sidewall of the retainer part or connector part is implemented as a through recess. In this way, and when the catch element is engaged with the recess it may be principally possible to insert a particularly designed tool into the recess from outside so as to urge the catch element out of the recess. The mutual connection between the retainer part and the connector could be then abrogated or released.

The catch element may comprise a protrusion and/or a beveled shape so as to deform or to move in a radial direction in the course of mutual assembly of the retainer part and the connector part. When reaching a final assembly configuration, e.g. when the distal end face of the connector sidewall engages the proximal end face of the retainer sidewall, the catch element may reach and/or overlap the recess and may relax into its initial state thereby engaging with the recess.

With some examples the catch element comprises a barb or hook shape to engage with the recess in order to provide a tension stable connection between the retainer part and the connector part.

With further examples there is provided a radial gap between an inside of the catch element and an outside of the medicament container or cartridge receiving space. The radial gap comprises a radial gap size larger than or equal to a moving distance required for the catch element to disengage from the recess. Insofar, and with a substantially tubular-shaped cartridge receiving space the inside of the retainer part or connector part may be somewhat elliptically-shaped or oval-shaped so as to provide a radial gap of predetermined size by way of which a radially inwardly directed deformation or movement of the catch element is allowed so as to provide a well-defined disengagement of the catch element and the recess and hence a respective disengagement of the fastening element and the complementary shaped counter fastening element of the proximal end and the distal connector.

According to a further example the distal connector comprises a predetermined breaking structure. The predetermined breaking structure may comprise a predetermined breaking point.

The predetermined breaking structure is configured to break apart the distal connector when a user attempts to disengage the distal connector from the proximal end of the retainer part.

In this way it can be ensured, that the cartridge holder comprising the retainer part and the connector part attached thereto can be disintegrated into the retainer part and the connector part.

But once the connector part has been disconnected from the retainer part, the distal connector breaks apart and is no longer usable to reattach to the retainer part. Here, a user of the injection device is obliged to dispose the connector part, e.g. fixedly attached or fixedly connected to the medicament container. Then, the user may have to use another container assembly, e.g. a preassembled arrangement of a new medicament container provided with a new connector part.

The new medicament container provided with the new connector part may be then inserted into the retainer part and the new connector part configured as a disposable component can be connected with the retainer part, implemented as a reusable component, thereby constituting a new cartridge holder.

The predetermined breaking structure may comprise a structural weakening, e.g. in a catch element or in or on a recess depending on the specific implementation of the fastening element or counter fastening element of the distal connector of the connector part.

According to a further example the connector part comprises a mechanical coding element to mechanically engage with a complementary shaped mechanical counter coding element of the device body. In this way, the connector part, e.g. undetachably fixed or connected with a dedicated medicament container, can be only used with an intended or dedicated device body or drive mechanism of an injection device. The mechanical coding element of the connector part prevents unintended cross use of a connector part provided with a medicament container not intended for use with the device body and/or the respective drive mechanism.

According to another aspect the present disclosure further relates to a cartridge assembly. The cartridge assembly comprises a cartridge holder as described above and further comprises a medicament container, e.g. in form of a cartridge, arranged inside the cartridge receiving space of the cartridge holder. Insofar, all features, effects and benefits as described above in connection with the cartridge holder equally apply to the cartridge assembly. With some examples the cartridge assembly comprises the medicament container fixedly arranged therein. The medicament container may be immovably fixed inside the cartridge receiving space as defined by the cartridge holder, which is e.g. formed by the retainer part connected with the connector part.

According to another aspect the present disclosure further relates to an injection device for setting and injecting of a dose of a medicament. The injection device comprises a device body, e.g. forming or constituting a proximal housing of the injection device. The injection device further comprises a drive mechanism arranged inside the device body. The drive mechanism is configured to operably engage with a medicament container containing the medicament. The drive mechanism typically comprises a piston rod to exert a distally directed driving force or expelling force onto a stopper of the medicament container so as to expel a dose of the medicament from the medicament container. The injection device further comprises one of a cartridge holder as described above and a cartridge assembly as described above, which is connectable or which is actually connected to a distal end of the device body. It should be noted that the injection device comprises a cartridge holder and/or a cartridge assembly as described above. Insofar, all features, effects and benefits as described above in connection with the cartridge holder and the cartridge assembly equally apply to the injection device.

According to a further aspect the present disclosure further relates to a kit or set of injection devices. The kit comprises a first injection device provided with a first device body, a first drive mechanism and one of a first cartridge holder and a first cartridge assembly. The kit further comprises a second injection device comprising a second device body, a second drive mechanism and one of a second cartridge holder and a second cartridge assembly. The first and second drive mechanisms may be differently configured. They may be suitable and intended for different types of medicament containers or medicaments.

The first and the second device bodies may distinguish from each other at least by a one of a counter fastening element and a counter coding element provided at or near a distal end of the device body. The counter fastening element is configured to mechanically engage with a complementary shaped fastening element of a matching cartridge holder. The counter coding element may be configured to mechanically engage with only a suitable coding element as provided at or near the proximal connector of the cartridge holder.

The first cartridge holder or cartridge assembly distinguishes from the second cartridge holder or second cartridge assembly by their first and second connector part. The first cartridge holder and the second cartridge holder, hence the first cartridge assembly and the second cartridge assembly may comprise a common and hence the same retainer part. Hence, for the first cartridge holder and/or the first cartridge assembly as well as for the second cartridge holder and the second cartridge assembly there may be provided one and the same retainer part comprising a proximal end configured to connect to either a distal connector of the second connector part and a distal connector of the second connector part. The first and second connector parts of first and second cartridge holders comprise equally shaped or equally configured distal connectors.

The first and second connector parts of the first and second cartridge holders and/or of first and second cartridge assemblies distinguish by their proximal connector and/or by a coding element configured to mechanically engage with a complementary-shaped counter coding element of the first and second device body.

In this way, the first cartridge holder of the first injection device can be only and exclusively used with the first device body and hence with the first drive mechanism of the first injection device. It cannot be connected or used with the second device body or second drive mechanism. Vice versa, the cartridge holder or second cartridge assembly can be exclusively used with the second device body and/or with the second drive mechanism. Unintended cross use, e.g. use of a first cartridge holder with the second device body or second drive mechanism as well as use of the second cartridge holder with the first device body or first drive mechanism is effectively prevented.

According to another aspect the present disclosure also relates to a container assembly for an injection device. The container assembly comprises a medicament container comprising a barrel filled with a medicament. The container assembly further comprises a connector part of a cartridge holder as described above. Here, the connector part is connected or fixed to the medicament container. It may be undetachably or removably fixed to the medicament container in a way as described above in relation to the container fastener of the connector part of the cartridge holder.

Insofar and since the container assembly uses a connector part of a cartridge holder as described above, all features, effects and benefits as described above in connection with the connector part of the cartridge holder equally apply to the container assembly. The container assembly may be preconfigured in that sense that the medicament container is preassembled with the connector part when commercially distributed to healthcare providers and/or patients. The container assembly may be implemented as a disposable assembly configured for use with a reusable retainer part of a cartridge holder and for use with a reusable device body and/or reusable drive mechanism of an injection device.

According to another aspect the present disclosure further relates to a container assembly kit or container assembly set. The container assembly kit comprises a first container assembly as described above. The first container assembly comprises a first medicament container and a first connector part connected or fixed to the first medicament container and comprising a first proximal connector and a first distal connector. The container assembly kit further comprises a second container assembly as described above. The second container assembly comprises a second medicament container and a second connector part connected or fixed to the second medicament container and comprising a second proximal connector and a second distal connector.

With the container assembly kit the first medicament container distinguishes from the second medicament container, e.g. by its geometric shape or size and/or by the medicament contained therein. The first proximal connector also distinguishes from the second proximal connector. In this way, it can be provided that only a first container assembly can be used with a first device body or first drive mechanism of an injection device and cannot be used with a differently configured second device body or second drive mechanism of another injection device.

According to a further example the first distal connector is substantially identical to the second distal connector. In this way, for the first container assembly as well as for the second container assembly there can be used one and the same retainer part to establish or to provide a cartridge holder. The retainer part can be connected to both, the first distal connector and the second distal connector, thereby forming or constituting a cartridge assembly comprising a cartridge holder as described above and comprising a medicament container arranged inside its cartridge receiving space. Such a cartridge holder can be then detachably connected with the distal end of a device body of an injection device.

In a further aspect the present disclosure relates to a method of assembling a cartridge assembly and/or an injection device as described above. Here and as a first step there are provided a medicament container, e.g. a cartridge, and a connector part of a cartridge holder, e.g. a cartridge holder as described above. Then, the medicament container is connected with the connector part. This way, there may be provided a container assembly as described above. In a subsequent step, the container assembly, hence the medicament container provided with the connector part is inserted into the retainer part of the cartridge holder. Here, the distal end of the medicament container is inserted in distal direction into the retainer part. Subsequently, and when reaching a final assembly position, the connector part and the retainer part are mutually connected. Typically, they are mutually assembled and attached to each other. Here, the distal connector of the connector part engages with the proximal end of the retainer part. A fastening element of the retainer part, e.g. provided on the retainer sidewall, engages with a complementary shaped counter fastening element of the distal connector of the connector part.

Thereafter and in a further step the cartridge assembly, i.e. the cartridge holder with the medicament container assembled therein is attached and connected to the device body thus forming or constituting the injection device. Here, and in particular, the connector part of the cartridge holder is connected with the device body, thereby forming or constituting the injection device.

Generally, the method of assembling the cartridge holder and/or the injection device is to be executed by a cartridge holder and an injection device as described above. Insofar all features, effects and benefits as described above in connection with any one of the cartridge holder, the cartridge assembly, the container assembly, the injection device and associated kits equally apply to the method of assembling the cartridge assembly and/or the injection device; and vice versa.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber.

In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, examples of a cartridge holder, a cartridge assembly, a container assembly and an injection device will be described in greater detail by making reference to the drawings, in which:

FIG. 1 shows an example of an injection device,

FIG. 2 shows a side view of one example of a cartridge holder,

FIG. 3 shows the cartridge holder of FIG. 2 in a perspective illustration,

FIG. 4 is an isolated view of the connector part of the cartridge holder,

FIG. 5 is a perspective illustration of a cartridge assembly,

FIG. 6 shows two examples of differently configured cartridge holders,

FIG. 7 is a longitudinal cross-section through a cartridge assembly,

FIG. 8 is a transverse cross-section through the cartridge assembly,

FIG. 9 is a cross-section through the cartridge assembly,

FIG. 10 is an enlarged longitudinal cross-section through the interface of the cartridge holder and the distal end of the device body,

FIG. 11 is a longitudinal cross-section through a cartridge assembly,

FIG. 12 is an enlarged illustration of the proximal end of the illustration of FIG. 11,

FIG. 13 is an enlarged view of the mutual connection between the retainer part and the connector part,

FIG. 14 is a further longitudinal cross-section through the cartridge assembly,

FIG. 15 is an enlarged cross-section of the proximal end of the cartridge assembly of FIG. 14,

FIG. 16 is an enlarged view of the interface between the retainer part and the connector part,

FIG. 17 is a partial illustration of the interface between the cartridge holder and the device body,

FIG. 18 is a flowchart of a method of assembling a cartridge assembly.

### DETAILED DESCRIPTION

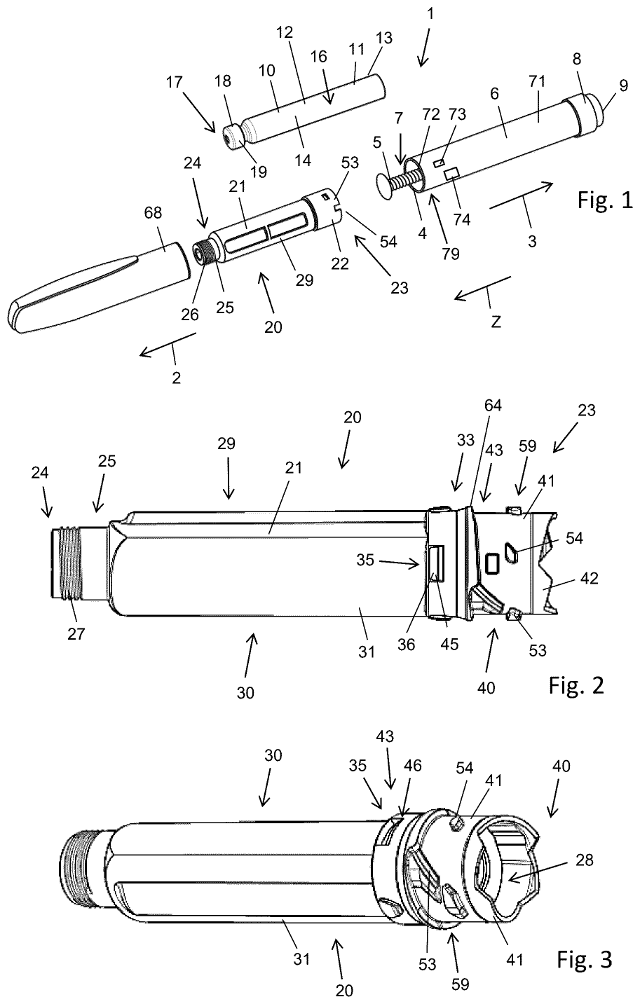

FIG. 1 shows an example of a drug delivery device implemented as an injection device 1, such as a pen-type injector. The drug delivery device 1 comprises numerous housing components. In detail, the drug delivery device 1 comprises a tubular shaped housing component implemented as a cartridge holder 20 sized to accommodate a medicament container 10. The injection device 1 further comprises a device body 6 forming or constituting a proximal housing component. The device body 6 houses a drive mechanism 7. The drive mechanism 7 may comprise a dose member 8 and/or a trigger 9 for setting and/or dispensing a dose of a medicament 16 contained in the medicament container 10, which in the present case may be implemented as a cartridge 11. Typically, the drive mechanism 7 comprises a piston rod 4, e.g. provided with a pressure piece 5 at its distal end. The pressure piece 5 is configured to abut in longitudinal direction (z) with a proximal end face of a piston 15 slidably arranged inside the barrel 12 of the cartridge 11. The piston 15 is for instance illustrated in FIG. 7.

The barrel 12, which may be of a vitreous material, such as glass, comprises a sidewall 13 with a main portion 14 of substantially tubular shape. The piston 15 is slidably displaceable along the main portion 14. Towards a dispensing end 17 the main portion 14 merges into a narrowing shoulder portion, which in turn merges in to a radially stepped down neck portion.

The injection device 1 further comprises a protective cap 68 configured to cover the dispensing end 24 of the injection device 1. The cartridge holder 20 comprises a distal end 24 and a proximal end 23. The distal end 24 is configured to engage with a needle assembly, e.g. a double-tipped injection needle, by way of which a dose of the liquid medicament 16 can be injected into biological tissue. For this, the distal end 24 of the cartridge holder 20 comprises a mechanical connector 25, e.g. implemented as a threaded section 27, which supports and allows for a mechanical, i.e. threaded connection, with a complementary shaped needle hub equipped with the injection needle.

The distal end 24 of the cartridge holder 20 further comprises an aperture 26 to receive a proximal portion or a proximally extending portion of the double-tipped injection needle. The injection needle is configured to pierce and/or to penetrate a seal 18 at the distal end of the cartridge 11.

The seal 18 may comprise a pierceable septum, which is fixed to a distally located head portion of the cartridge 11 by way of a septum retainer 19. The septum retainer 19 may comprise a beaded cap, e.g. made of a pliable material, such as aluminum or plastic.

The septum retainer 19 comprises an aperture that aligns with the aperture 26 when the cartridge 11 is received in the interior of the cartridge holder 20. Inside the cartridge holder 20 there is provided a cartridge receiving space 28, which is effectively occupied by the cartridge 11 when assembled inside the cartridge holder 20. The cartridge holder 20 comprises a proximal end 23, which may be provided with an insert 42 sized and configured for insertion into a receptacle 72 as provided at the distal end of the device body 6. Towards the distal direction 2, the insert 42 is delimited by a flange portion 64. When the cartridge holder 20 is attached to the device body 6 this forming or constituting the injection deice 1, the flange portion 64 may be in longitudinal abutment with a distal end face of the sidewall 71 of the receptacle 72 or device body 6.

On the inside of the receptacle 72 there may be provided a counter fastening element 73, e.g. in form of a radial protrusion or radial recess to engage with a complementary shaped fastening element 53 provided on an outside surface of the insert 42. This way, the proximal end of the cartridge holder 20 can be fixed and attached to the body 6.

Optionally, the proximal end 23 of the cartridge holder 20 comprises a coding element 54 by way of which the cartridge holder 20 and/or a cartridge assembly 100 can be mechanically encoded to distinguish from another cartridge holder 20′ or cartridge assembly 100′, e.g. equipped with a different type of cartridge 11 or with a cartridge containing a different medicament 16. The coding element 54 or coding feature may be complementary shaped or complementary positioned to a counter coding element 74 as provided on or inside the device body 6. A non-matching pair of a coding element 54 and a counter coding element 74 may prevent a mutual fastening of the cartridge holder 20 to the body 6.

A mutual fastening and fixing of the cartridge holder 20 to the device body 6 may be only provided and allowed when the coding element 54 of the cartridge holder 20 matches in size, shape and position with the corresponding counter coding element 74 as provided on or inside the device body 6.

In FIGS. 2 and 3 the cartridge holder 20 of the injection device 1 is illustrated in greater detail. The cartridge holder 20 comprises a sidewall 21 of substantially tubular shape. The cartridge holder 20 comprises a proximal connecting end 22 for interconnecting with a distal end 79 of the device body 6. The cartridge holder 20 comprises an elongated body 29 extending in longitudinal direction (z). Towards the distal end 24 the elongated body 29 comprises the connector 25. Towards the proximal end the body 29 comprises the connecting end 22 for detachably interconnecting with the device body 6.

As it becomes particularly apparent from FIGS. 2-5 the cartridge holder 20 comprises a retainer part 30 and a connector part 40. The connector part 40 forms the proximal end 23 of the cartridge holder 20. The retainer part 30 forms or constitutes the distal end 24. The retainer part 30 comprises a retainer sidewall 31 confining a distal portion of the cartridge receiving space 28. The connector part 40 comprises a connector sidewall 41. As illustrated in FIG. 2 the connector sidewall 41 and the retainer sidewall 31 are substantially flush as seen in longitudinal direction.

Insofar, the connector sidewall 41 is or forms a longitudinal extension of the retainer sidewall 31 when the connector part 40 is connected to the retainer part 30. In the interface between the retainer part 30 and the connector part 40 there may be provided a radially outwardly protruding annular flange portion 64.

The flange portion 64 may be provided at or near a distal end of the connector part 40. The flange portion 64 may comprise a proximally facing abutment face, e.g. engaging with an end face of the sidewall 71 of the device body 6.

The retainer part 30 comprises a proximal end 33, which is configured to engage with the connector part 40. The connector part 40 comprises a distal connector 43 for establishing a mechanical connection with the proximal end 33 of the retainer part 30. The distal connector 43 comprises two counter fastening element 45 each of which implemented as a catch element 46 featuring a radially outwardly extending protrusion 47 at a distal end of a longitudinal extension that protrudes distally from a distally facing end face 44 of the connector sidewall 41. The counter fastening elements 45 are configured to engage with the fastening elements 35 as provided on or in the proximal end 33.

The catch element 46 comprises a beveled edge 48 facing radially outwardly and in distal direction 2. For mutually interconnecting the retainer part 30 and the connector part 40 the counter fastening element 45 engages the proximally facing end face of 34 of the retainer sidewall 31 in the course of inserting the counter fastening element 45 into the open ended proximal end of the retainer sidewall 31.

This way, the counter fastening element 45 and hence the catch element 46 is elastically deformed radially inwardly to allow and support a distally directed insertion thereof into a receptacle 32 as provided at the proximal end 33. When reaching a final assembly configuration, e.g. in which the end face 34 of the retainer sidewall 31 gets in longitudinal abutment with the complementary shaped end face 44 of the connector sidewall 41 the protrusion 47 of the catch element 46 engages and snaps into a recess 36 of the fastening element 35, thereby locking the connector part 40 in position on the retainer part 30.

This type of connection is tension stable as well as pressure stable.

The recess 36 is implemented as a through recess. By making use of a particularly shaped tool shaped and designed for insertion into the recess 36 from outside, the protrusion 46 and hence the catch element 46 can be urged radially inwardly thereby disengaging from the recess 36 to thereby disengage the counter fastening element 45 of the connector part 40 from the fastening element 35 of the retainer part 30.

Insofar, the connector part 40 and the retainer part 30 can be detachably connected. The connector part 40, in particular a connector sidewall 41, protrudes in proximal direction 3 from the retainer sidewall 31 when the connector part 40 is attached and fixed to the retainer part 30. The connector sidewall 41 forms an insert 42 for insertion into a receptacle 72 of the body 6. On the inside of the sidewall 71 of the receptacle 72 there is provided at least one counter fastening element 73 configured to engage with a fastening element 53 as provided on the outside surface of the connector sidewall 41. Here, the fastening element 53 may comprise one of a protrusion and a recess.

As illustrated in FIGS. 2 and 3 the fastening element 53 comprises a fastening protrusion 55. The fastening element 53 and the counter fastening element 73 may be configured to provide various types of detachable or releasable mechanical interconnections. The fastening element 53 and the counter fastening element 73 may be configured to provide one of a bayonet joint, a threaded joint or a clip joint.

The connector part 40 further comprises the coding element 54. Also, the coding element 54 is provided on the outside surface of the connector sidewall 41. It is configured to mechanically engage with the complementary-shaped counter coding element 74 as provided on an inside surface of the sidewall 71 of the receptacle 72 of the device body 6. It comprises a coding protrusion 56 to match and/or to engage with a complementary shaped coding recess of the counter coding element 74 of the device body 6. The coding element 54 may be encoded by way of its geometric structure and/or by an arrangement and position or orientation relative to the fastening element 53.

Moreover, also the proximal end face 60 of the connector sidewall 41 may be provided with an uneven profile as seen in circumferential and longitudinal direction. Here, a profiled, saw tooth-like, rippled or undulated structure of the proximal end face 60 of the connector sidewall 41 may equally serve and provide a mechanical coding to engage with a complementary shaped mechanical counter coding as provided on the inside of the receptacle 72 of the sidewall 71 of the device 46.

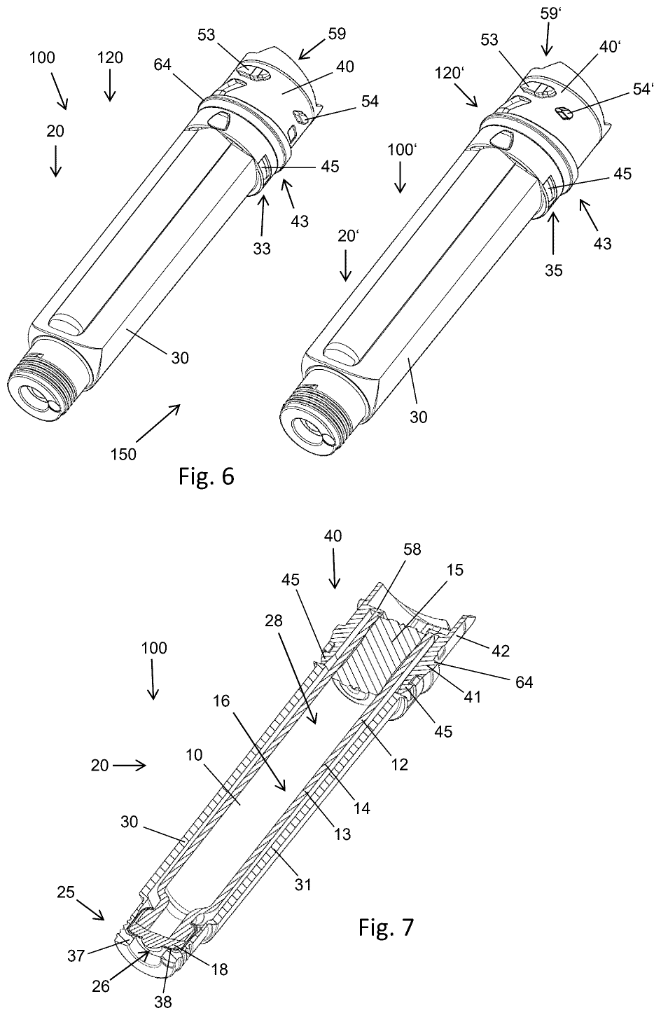

Two examples of different coding elements 54, 54′ are apparent from FIG. 6. On the left side of FIG. 6 there is illustrated a first cartridge assembly 100 comprising a cartridge holder 20 and a cartridge 11 or medicament container 10 arranged therein. The cartridge assembly 100 comprises the retainer part 30 connected with a container assembly 120. The container assembly 120 comprises the connector part 40 and the medicament container 10.

The first cartridge assembly 100 and hence the first cartridge holder 20 comprises a first connector part 40 featuring a first fastening element 53 and a first coding element 54. The first connector part 40 comprises a first distal connector 43 for interconnecting with the proximal end 33 of the retainer part 30.

On the right side of FIG. 6 there is illustrated a second cartridge holder 20′ forming a second cartridge assembly 100′. The second cartridge assembly 100′ comprises a second container assembly 120′. The second container assembly 120′ comprises a second connector part 40′ and a second medicament container 10 (not illustrated). The second medicament container may distinguish from the first medicament container, either by way of its size, its geometric shape and/or by the type and/or concentration of the medicament located therein.

The first and second container assemblies 120, 120′ form or constitute a kit of container assemblies 150.

Accordingly, the second connector part 40′ distinguishes from the first connector part 40. As illustrated, the coding element 54′ of the second connector part 40′ is at a different position relative to the fastening element 53 compared to the position of the coding element 54 relative to the fastening element 52 of the first connector part 40. Accordingly, a proximal connector 59′ of the second connector part 40′ can be only or exclusively connected to a second type of a device body 6 whereas the first connector part 40 equipped with the first proximal connector 59 can be only and exclusively connected to a first type of a device body 6. The first and the second device bodies distinguish from each other by their counter coding elements 74.

Here, the counter coding element of the second device body is provided at a different relative position to the counter fastening element 73 compared to the counter coding element 74 and the counter fastening element 73 of the first device body 6.

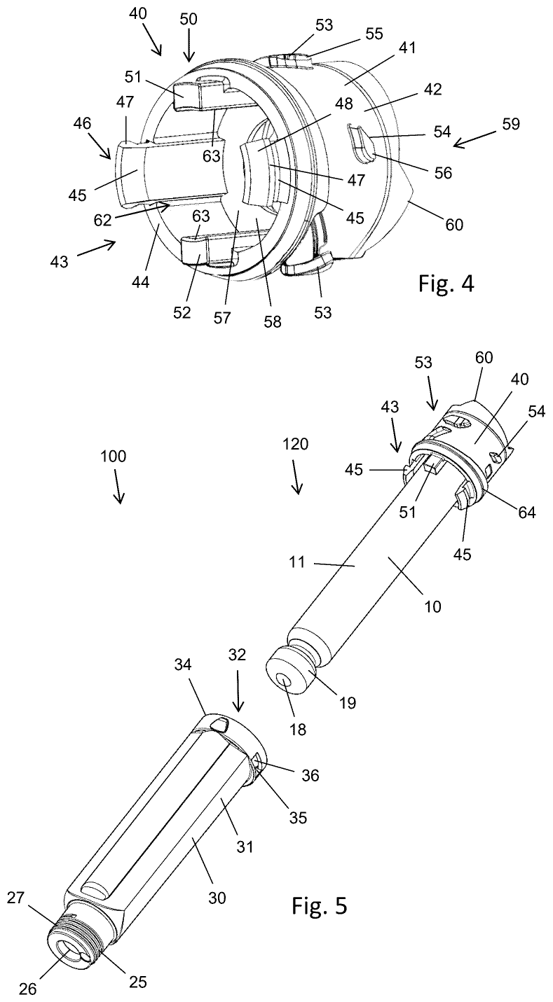

As it is further apparent from FIGS. 4, 5, 8 and 9 the connector part 40 comprises a receptacle 62 sized and shaped to receive a proximal end of the medicament container 10. Here, the connector sidewall 41 confines the receptacle 62 in circumferential direction. The receptacle 62 is delimited in proximal and hence longitudinal direction by a stop face 57 as provided on a flange portion 58 protruding radially inwardly from an inside of the connector sidewall 41. As it is for instance apparent from FIGS. 7 and 10 the radially inwardly extending flange portion 58 with its distally facing stop face 57 is configured to longitudinally abut with the proximal end of the barrel 12 and hence with the sidewall 13 of the cartridge 11 or medicament container 10.

Towards the distal end the medicament container 10 may be in longitudinal abutment with an end wall 37 of the retainer part 30. The end wall 37 may form the distal end 24 of the cartridge holder 20. The end wall 37 may extend radially inwardly from the cylindrical portion of the connector 25. The end wall 37 may comprise a radially inwardly extending flange section featuring a proximally facing stop face 38. The stop face 38 may get in longitudinal abutment with the distal end of the medicament container 10, e.g. with the seal 18 or septum retainer 19 as illustrated in FIG. 7, 11 or 14.

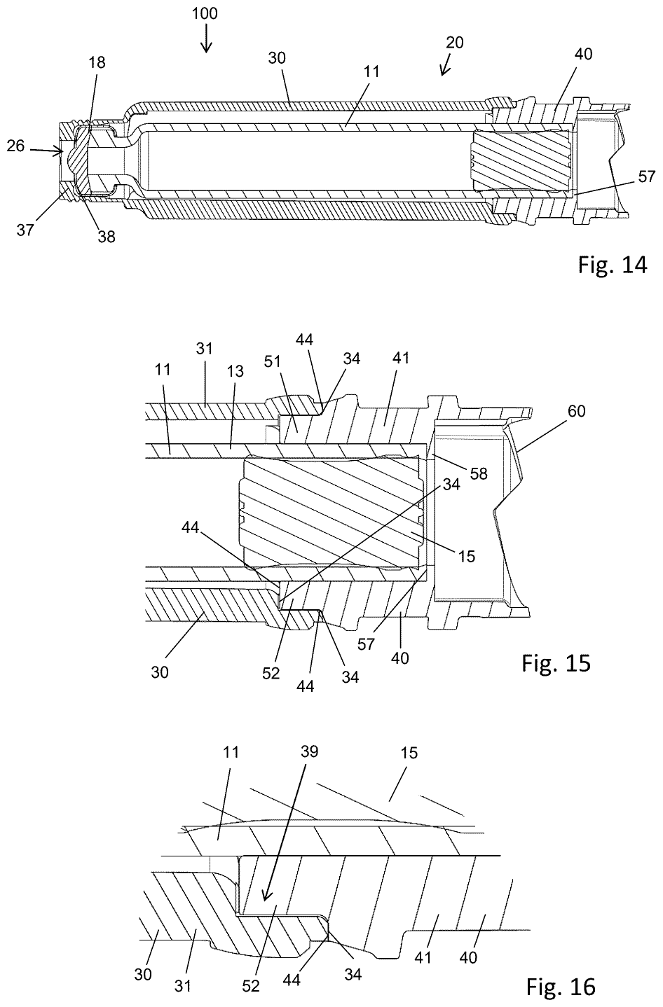

As it is apparent from FIGS. 7, 11 and 14 the medicament container 10 may be longitudinally secured inside the cartridge receiving space 28. It may be in a longitudinal distal abutment with the end wall 37 and hence with the proximally facing stop face 38. It may be further in longitudinal abutment with the distally facing stop face 57 of the flange portion 58 of the connector part 40. The mutual connection between the connector part 40 and the retainer part 30 is tension stable and pressure stable. In this way and when reaching a final assembly configuration, in which the fastening element 35 engages the counter fastening element 45 and hence when the catch element 46 engages the recess 36, the medicament container 10 may be clamped or at least securely retained inside the cartridge receiving space with regard to the longitudinal direction (z).

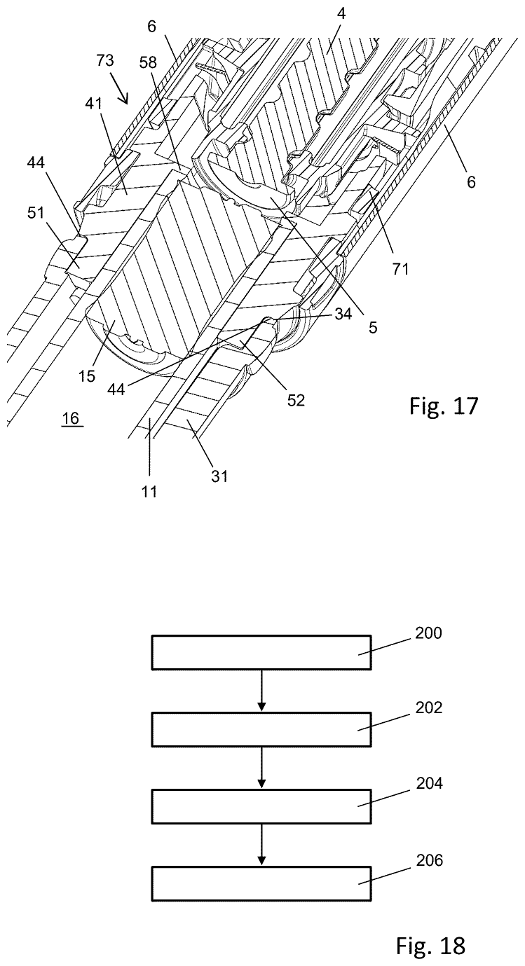

As it is further illustrated in FIGS. 4 and 14-17 the connector part 40 further comprises a container fastener 50. The container fastener 50 comprises a first fastening rib 51 and a second fastening rib 52. Like the counter fastening elements 45 also the fastening ribs 51, 52 are located on opposite sides on the inside of the connector sidewall 41. As further illustrated in FIG. 4 the fastening ribs 51, 52 may protrude in distal direction 2 from the annular shaped end face 44 of the connector sidewall 41. They also extend radially inwardly from the tubular shaped connector sidewall 41. They may protrude radially inwardly into the cartridge receiving space 28.

As it is particularly apparent from FIG. 9 the radial distance between oppositely located first and second fastening rib 51, 52 is smaller than a radial distance between the oppositely located counter fastening element 45 and hence smaller than the radial gap between the oppositely located catch elements 46. The radial distance between the first and the second fastening ribs 51, 52 equally matches or is even slightly smaller than an outside distance or outside diameter of the barrel 12 at its proximal end. In this way the barrel 12 of the medicament container 10 can be fitted into the receptacle 62 thereby engaging with the inside facing engaging surfaces 63 of the first and second fastening ribs 51, 52.

Here, the outside surface of the sidewall 13 of the barrel 12 may frictionally engage with the inside surface, hence the engaging surface 63 of the first and second fastening ribs 51, 52. In this way, the medicament container 10 can be preassembled and fastened to the connector part 40, thereby forming a container assembly 120 as e.g. illustrated in FIG. 5. The friction fit between the container fastener 50 and the barrel 12 may be such that a consumer or patient is not able to exert a force large enough to detach the medicament container 10 from the container fastener. A pull-off force may be larger than what an average person would be able to apply.

Hence, the container fastener 50 may be in a press fitting configuration with the medicament container 10. With some examples, the container assembly, i.e. the medicament container 10 prefixed with the connector part 40 may be commercially distributed as a disposable unit. Accordingly, the retainer part 30 detachably connectable to the connector part 40 can be implemented as a reusable component.

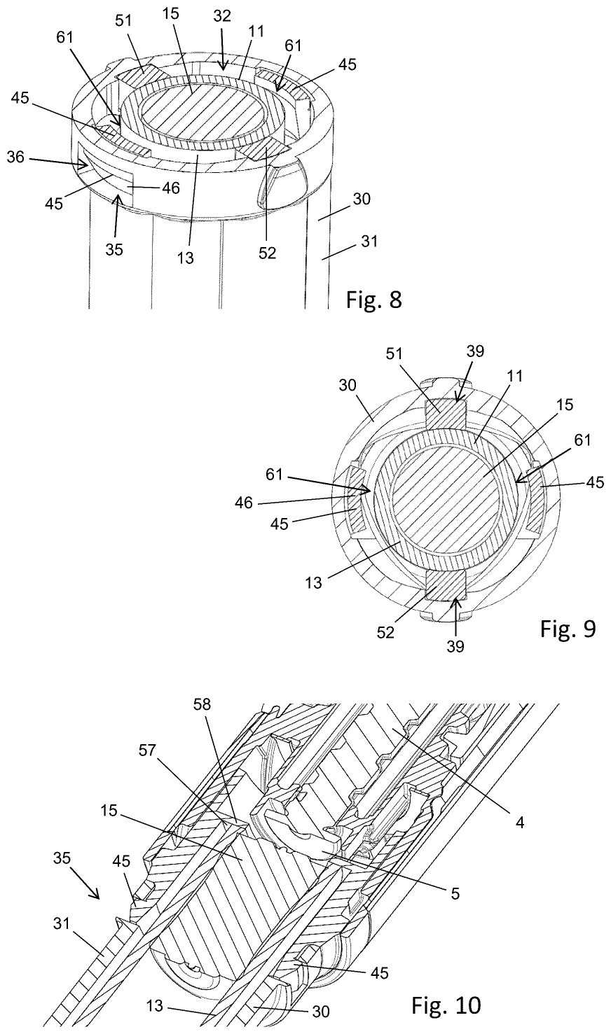

As it is particularly illustrated in FIGS. 8 and 9 there is provided a radial gap 61 between an inside of the counter fastening element 45 and an outside of the sidewall 13 of the barrel 12 of the medicament container 10. The radial size of this gap 61 is typically slightly larger than a radial distance required for disengaging the catch element 46 from the recess 36. In this way, and with a cartridge 11 or medicament container 10 assembled inside the cartridge holder 20 it is generally possible to urge the protrusions 47 and hence the catch elements 46 radially inwardly to such an extent that the protrusion 47 disengages from the recess 36, thereby disengaging and releasing the connector 43 from the fastening element 35 of the proximal end 33 of the retainer part 30.

Typically, and as particularly illustrated in FIG. 13 the catch elements 46 are provided with a predetermined breaking structure 49, which is implemented as a structural weakening. By urging the protrusion 46 and hence the catch elements 46 radially inwardly, e.g. by inserting a appropriately sized tool into the recess 36 from outside, the nose portion or protrusion 47 of the catch element 46 may break and disintegrate, thus destroying and/or disabling the function of the counter fastening element 45.

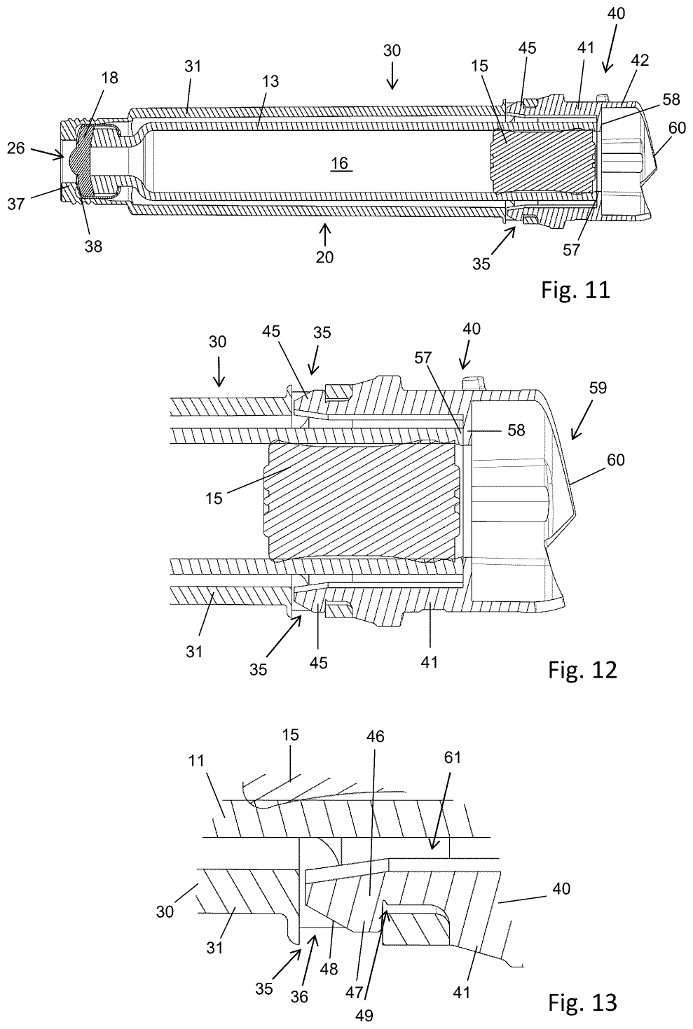

Once the catch element 46 has been transferred from the fastening configuration as e.g. illustrated in FIGS. 11-13 into a release configuration, e.g. by urging the catch element 46 radially inwardly, the catch element 46 disintegrates into at least two pieces and becomes substantially inoperable.

In this way the connector part 40, which may be still connected and fastened to the medicament container 10 cannot be used any longer. The medicament container 10 together with the connector part 40 is then to be discarded in its entirety. A user then has to be provided with a new container assembly 120, i.e. a new medicament container 10 attached and provided with a new connector part 40. The connector part 40 and the medicament container 10 can be provided preassembled. They have to be connected with the reusable retainer part 30 thus forming or constituting a new or refurbished cartridge holder 20. The new cartridge holder 20, i.e. provided with the new cartridge 11 and the new connector part 40 can then be reattached to the reusable device body 6 and hence to the reusable drive mechanism 7 in a conventional manner.

The proximal end 33 of the retainer part 30 comprises a kind of a receptacle 32 sized and shaped to receive the fastening ribs 51, 52 and the counter fastening element 45 of the connector part 40. For a correct alignment of the connector part 40 with the retainer part 30 the receptacle 32 as provided at the proximal end of the retainer sidewall 31 comprises at least one recess 39, e.g. implemented as a longitudinal groove configured and sized to receive the fastening rib 51 as illustrated in FIG. 9. Here, the groove-like recess 39 provides a mechanical guiding function for the longitudinally extending fastening ribs 51, 52.

The fastening ribs 51, 52, which may protrude in longitudinal direction from the counter fastening element 45 can be aligned with the complementary shaped grooves or recesses 39 on the inside of the retainer sidewall 31, thereby providing a mounting aid for inserting the fastening ribs 51, 52 and hence the counter fastening element 45 into the receptacle 32 until a final assembly configuration has been reached.

Insofar, the fastening ribs 51, 52 provide a twofold or double function. First of all, they provide a fastening of the medicament container 10 to the connector part 40. Second, they enable and facilitate a proper alignment and engagement of the distal connector 43 with the fastening element 35 in the course of insertion into the receptacle 32 of the proximal end 33 of the retainer part 30.

In FIG. 18 a flowchart of a method of assembling an injection device 1 or of interchanging a medicament container 10 is illustrated. In a first step 200 a medicament container 10, e.g. a cartridge 11, is connected with a connector part 40. Here, the connector part 40 is provided as a separate component. The container fastener 50 of the connector part 40 receives the proximal end of the medicament container 10. Here, the proximal end of the barrel 12 may be clamped or frictionally engaged with diametrically oppositely located fastening ribs 51, 52.

In a subsequent step 202 the container assembly 120 formed by the medicament container 10 and the connector part 40 is inserted into the retainer part 30. Here, the distal portion or main portion of the medicament container 10 protruding distally from the connector part 40 is inserted in longitudinal distal direction 2 through the receptacle 32 and into the interior of the retainer part 30. In the subsequent step 204 there is established a mechanical connection between the proximal end 33 of the retainer part 30 and the distal connector 43 of the connector part 40. After this step there is provided a cartridge assembly 100, i.e. a cartridge holder 20 readily equipped with a medicament container 10. In a subsequent step 206 the cartridge holder 20 and hence the cartridge assembly 100 is connected to the distal end 79 of the device body 46, thereby forming or constituting the injection device 1, which is then ready for setting and dispensing of one or several doses of the medicament 16. For this the proximal connector 59 of the connector part 40 is connected and/or fixed to the distal end 79, and in particular to the counter fastening element 73 of the device body 6.

### REFERENCE NUMBERS

1 injection device
2 distal direction
3 proximal direction
4 piston rod
5 pressure piece
6 device body
7 drive mechanism
8 dose member
9 trigger
10 medicament container
11 cartridge
12 barrel
13 sidewall
14 main portion
15 piston
16 medicament
17 dispensing end
18 seal
19 septum retainer
20 cartridge holder
21 sidewall
22 connecting end
23 proximal end
24 distal end
25 connector
26 aperture
27 threaded section
28 cartridge receiving space
29 body
30 retainer part
31 retainer sidewall
32 receptacle
33 proximal end
34 end face
35 fastening element
36 recess
37 end wall
38 stop face
39 recess
40 connector part
41 connector sidewall
42 insert
43 distal connector
44 end face
45 counter fastening element
46 catch element
47 protrusion
48 beveled edge
49 predetermined breaking structure
50 container fastener
51 fastening rib
52 fastening rib
53 fastening element
54 coding element
55 fastening protrusion
56 coding protrusion
57 stop face
58 flange portion
59 proximal connector
60 end face
61 gap
62 receptacle
63 engaging surface
64 flange portion
68 protective cap
71 sidewall
72 receptacle
73 counter fastening element
74 counter coding element
79 distal end
100 cartridge assembly
120 container assembly
150 container assembly kit

1. A cartridge holder for an injection device, the cartridge holder comprising:
an elongated body extending in a longitudinal direction (z), the elongated body comprises a distal end and a proximal end opposite to the distal end, wherein the elongated body confines a cartridge receiving space to accommodate a medicament container,
a retainer part comprising a retainer sidewall confining a distal portion of the cartridge receiving space, the retainer part comprising a proximal end,
a connector part comprising a connector sidewall and a distal connector configured to connect to the proximal end of the retainer part and comprising a proximal connector configured to connect to a distal end of a device body of the injection device, wherein the device body is configured to accommodate a drive mechanism to operably engage with the medicament container.

2. The cartridge holder according to claim 1, wherein the connector sidewall forms a longitudinal extension of the retainer sidewall when the connector part is connected to the retainer part.

3. The cartridge holder according to claim 2, wherein the connector sidewall and the retainer sidewall flush in the longitudinal direction (z).

4. The cartridge holder according to claim 2, wherein an inside surface of the connector sidewall flushes with an inside surface of the retainer sidewall and/or wherein an outside surface of the connector sidewall with an outside surface of the retainer sidewall.

5. The cartridge holder according to claim 1, wherein the connector part comprises a flange portion extending radially inwardly from the connector sidewall, the flange portion comprising a distally facing stop face configured to abut with a proximal end of the medicament container.

6. The cartridge holder according to claim 1, wherein the proximal end of the retainer part comprises a proximal end face complementary shaped to a distal end face of the connector part and wherein the proximal end face is in longitudinal abutment with the distal end face when the connector part is connected to the retainer part.

7. The cartridge holder according to claim 1, wherein the distal connector of the connector part and the proximal end of the retainer part are configured to form at least one of a tension stable and a pressure stable mechanical connection.

8. The cartridge holder according to claim 1, wherein the connector part comprises a container fastener configured to mechanically engage with a proximal end of the medicament container.

9. The cartridge holder according to claim 8, wherein the container fastener is configured to non-detachably connect to the medicament container.

10. The cartridge holder according to claim 8, wherein the container fastener comprises a first fastening rib and a second fastening rib on opposite sides of the cartridge receiving space, wherein the first fastening rib and the second fastening rib are configured to frictionally engage with a barrel of the medicament container.

11. The cartridge holder according to claim 10, wherein at least one of the first fastening rib and the second fastening rib protrudes distally from a distal end face of the connector part and engages with a complementary-shaped recess of the retainer part when the connector part is connected to the retainer part.

12. The cartridge holder according to claim 1, wherein one of the proximal end and the distal connector comprises a fastening element complementary shaped to a counter fastening element of the other one of the proximal end and the distal connector.

13. The cartridge holder according to claim 12, wherein one of the fastening element and the counter fastening element comprises a recess and wherein the other one of the fastening element and the counter fastening element comprises a catch element configured to engage with the recess.

14. The cartridge holder according to claim 13, wherein the catch element of one of the retainer part and the connector part extends in longitudinal direction and is configured to engage with the recess provided in the connector sidewall or retainer sidewall of the other one of the retainer part and the connector part.

15. The cartridge holder according to claim 1, wherein the distal connector comprises a predetermined breaking structure, which is configured to break apart the distal connector when a user attempts to disengage the distal connector from the proximal end of the retainer part.

16. The cartridge holder according to claim 1, wherein the connector part comprises a mechanical coding element to mechanically engage with a complementary shaped mechanical counter coding element of the device body.

17. A cartridge assembly comprising a cartridge holder according to claim 1 and further comprising a medicament container arranged inside the cartridge receiving space.

18. An injection device for setting and injecting of a dose of a medicament, the injection device comprises:
a device body,
a drive mechanism arranged inside the device body, wherein the drive mechanism is configured to operably engage with a medicament container containing the medicament, and
one of a cartridge holder according to claim 1 and a cartridge assembly connectable or connected to a distal end of the device body.

19. A container assembly for an injection device, the container assembly comprising:
a medicament container comprising a barrel filled with a medicament, and
a connector part of a cartridge holder according to claim 1, wherein the connector part is connected or fixed to the medicament container.

20. A container assembly kit comprising:
a first container assembly according to claim 19, the first container assembly comprising a first medicament container and a first connector part connected or fixed to the first medicament container and comprising a first proximal connector and a first distal connector,
a second container assembly according to claim 19, the second container assembly comprising a second medicament container and a second connector part connected or fixed to the second medicament container and comprising a second proximal connector and a second distal connector,
wherein the first medicament container distinguishes from the second medicament container and wherein the first proximal connector distinguishes from the second proximal connector.
